(19) United States
(12) Patent Application Publication
(10) Pub. No.: US 2010/0070013 A1
(43) Pub. Date: Mar. 18, 2010

(54) Medical Device With Microsphere Drug Delivery System

(75) Inventor: Eunsung Park, Plymouth, MN (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/212,828

(22) Filed: Sep. 18, 2008

(51) Int. Cl. A61F 2/84; A61F 2/82

(52) U.S. Cl. 623/1.11; 623/1.42

(57) ABSTRACT

A system for treating a vascular condition includes a therapeutic agent eluting medical device having a multilayered coating comprising microspheres of variable wall thicknesses. The wall thicknesses and the composition of the microspheres provide a controlled delivery system for one or more therapeutic agents. Another embodiment of the invention includes a method of treating a vascular condition by placing a stent at the treatment site and delivering one or more therapeutic agents from a coating on at least a portion of the stent surface. The coating comprises microspheres of variable wall thicknesses and optionally, an agent that modulates the rate of degradation of the microspheres.

### TECHNICAL FIELD

This invention relates generally to biomedical devices that are used for treating vascular conditions. More specifically, the invention relates to a therapeutic agent eluting system having a multilayered coating comprising microspheres of variable wall thicknesses disposed on the surface of a stent or balloon.

### BACKGROUND OF THE INVENTION

Stents are generally cylindrical-shaped devices that are radially expandable to hold open a segment of a vessel or other anatomical lumen after implantation into the body lumen.

Various types of stents are in use, including expandable and self-expanding stents. Expandable stents generally are conveyed to the area to be treated on balloon catheters or other expandable devices. For insertion into the body, the stent is positioned in a compressed configuration on the delivery device. For example, the stent may be crimped onto a balloon that is folded or otherwise wrapped about the distal portion of a catheter body that is part of the delivery device. After the stent is positioned across the lesion, the balloon is expanded by the delivery device, causing the diameter of the stent to expand. For a self-expanding stent, commonly a sheath covering the stent is retracted, allowing the unconstrained stent to expand.

Stents are used in conjunction with balloon catheters in a variety of medical therapeutic applications, including intravascular angioplasty to treat a lesion such as plaque or thrombus. For example, a balloon catheter device is inflated during percutaneous transluminal coronary angioplasty (PTCA) to dilate a stenotic blood vessel. When inflated, the pressurized balloon exerts a compressive force on the lesion, thereby increasing the inner diameter of the affected vessel. The increased interior vessel diameter facilitates improved blood flow. Soon after the procedure, however, a significant proportion of treated vessels restenose.

To reduce restenosis, stents, constructed of metals or polymers, are implanted within the vessel to maintain lumen size. The stent is sufficiently longitudinally flexible so that it can be transported through the cardiovascular system. In addition, the stent requires sufficient radial strength to enable it to act as a scaffold and support the lumen wall in a circular, open configuration. Configurations of stents include a helical coil, and a cylindrical sleeve defined by a mesh, which may be supported by a stent framework of struts or a series of rings fastened together by linear connecter portions.

Stent insertion may cause undesirable reactions such as inflammation resulting from a foreign body reaction, infection, thrombosis, and proliferation of cell growth that occludes the blood vessel. Stents capable of delivering one or more therapeutic agents have been used to treat the damaged vessel and reduce the incidence of deleterious conditions including thrombosis and restenosis.

Polymer coatings applied to the surface of stents and/or balloons have been used to deliver drugs or other therapeutic agents at the placement site of the stent. The coating is a thin polymeric layer applied to the surface of the stent framework, so that the stent has a low profile. However, the amount of therapeutic agent that can be delivered, and the time period of release are frequently limited by the dimensions of the coating.

It is desirable, therefore, to provide an implantable stent having a delivery system for one or more therapeutic agents that overcomes many of the limitations and disadvantages of the stents described above.

### SUMMARY OF THE INVENTION

One aspect of the present invention provides a system for treating a vascular condition comprising a catheter, a medical device disposed on the catheter, a therapeutic agent delivery coating disposed on at least a portion of the medical device, and at least one therapeutic agent. The coating comprises a first plurality of biodegradable microspheres having a first wall thickness, and a second plurality of biodegradable microspheres having a second wall thickness. The release rate of the therapeutic agent is determined by the first and second wall thicknesses of the microspheres.

Another aspect of the invention provides a medical device for treating a vascular condition including a coating disposed on a surface of the device. The coating comprises a plurality of biodegradable microspheres of variable wall thicknesses. The rate of release of at least one therapeutic agent from the coating is dependent on the wall thicknesses of the biodegradable microspheres.

The present invention is illustrated by the accompanying drawings of various embodiments and the detailed description given below. The drawings should not be taken to limit the invention to the specific embodiments, but are for explanation and understanding. The detailed description and drawings are merely illustrative of the invention rather than limiting, the scope of the invention being defined by the appended claims and equivalents thereof. The drawings are not to scale. The foregoing aspects and other attendant advantages of the present invention will become more readily appreciated by the detailed description taken in conjunction with the accompanying drawings.

### BRIEF DESCRIPTION OF THE DRAWINGS

FIG. 1 is a schematic illustration of a system for treating a vascular condition including a therapeutic agent carrying stent coupled to a catheter, in accordance with one embodiment of the present invention;

FIG. 2 is a schematic illustration of a portion of a medical device surface coated with layers of microspheres having variable wall thicknesses providing therapeutic agent release at a constant rate, in accordance with the present invention;

FIG. 3 is a schematic illustration of a portion of a medical device surface coated with microspheres having variable wall thicknesses and differing in composition, in accordance with the present invention;

FIG. 4 is a schematic illustration of a portion of a medical device surface coated with microspheres having variable wall thicknesses, and including an agent that facilitates the dissolution of the microspheres, in accordance with the present invention; and

FIG. 5 is a flow diagram for a method of treating a vascular condition with a stent having a coating comprising microspheres of variable wall thicknesses and a therapeutic agent disposed within the microspheres, in accordance with the present invention.

### DETAILED DESCRIPTION

Throughout this specification, like numbers refer to like structures.

The present invention is directed to a system for treating abnormalities of the cardiovascular system comprising a catheter and a therapeutic agent-carrying stent disposed on the catheter. In one embodiment, the stent has a coating comprising microspheres having variable wall thicknesses and at least one therapeutic agent disposed within the coating that is eluted at a controlled rate at a treatment site. Though the invention is described in relation to a stent or a balloon, the invention may be practiced on other therapeutic and interventional devices such as tracheal stents, stent grafts, and other grafts.

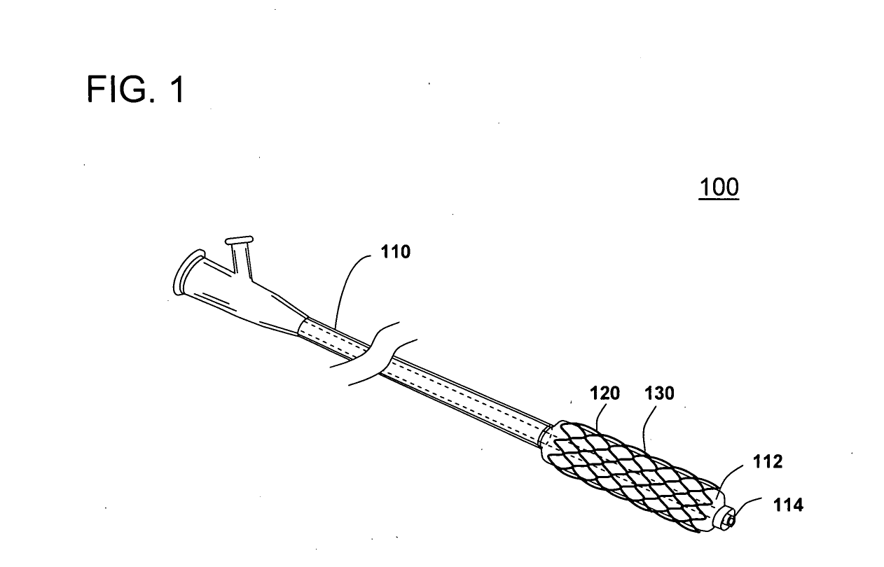

FIG. 1 shows an illustration of a system 100 for treating a vascular condition, comprising stent 120 coupled to catheter 110, in accordance with one embodiment of the present invention. In an exemplary embodiment, catheter 110 includes a balloon 112 that expands and deploys stent 120 within a vessel of the body. After positioning stent 120 within the vessel with the assistance of a guide wire traversing through guide wire lumen 114 inside catheter 110, balloon 112 is inflated by pressurizing a fluid such as a contrast fluid or saline solution that fills a tube inside catheter 110 and balloon 112. Stent 120 is expanded until a desired diameter is reached; then the fluid is depressurized or pumped out, separating balloon 112 from stent 120 and leaving stent 120 deployed in the vessel of the body. Alternately, catheter 110 may include a sheath that retracts to allow expansion of a self-expanding version of therapeutic agent carrying stent 120. In various embodiments of the invention, a surface of the balloon, the stent, or both is covered with a coating that delivers at least one therapeutic agent.

Therapeutic agent carrying stent 120 includes a stent framework 130. In one embodiment of the invention, stent framework 130 comprises struts that form a mesh and provide a porous stent wall. In one embodiment, stent framework 130 comprises one or more of a variety of biocompatible metals such as stainless steel, titanium, magnesium, chromium, cobalt, nickel, gold, iron, iridium, chromium/titanium alloys, chromium/nickel alloys, platinum/iridium alloys, chromium/cobalt alloys, such as MP35N and L605, cobalt/titanium alloys, nickel/titanium alloys, such as nitinol, platinum, and platinum-tungsten alloys. In another embodiment, stent framework 130 comprises one or more biocompatible thermoplastic polymers such as polyethylene, polypropylene, polymethyl methacrylate, polycarbonate, polyesters, polyamides, polyurethanes, polytetrafluoroethylene (PTFE), polyvinyl alcohol, silicone, polyether-amide elastomers, a combination of oligo(e-caprolactone)diol and crystallizable oligo(r-dioxanone)diol, other shape memory polymers, and other suitable polymers and combinations thereof.

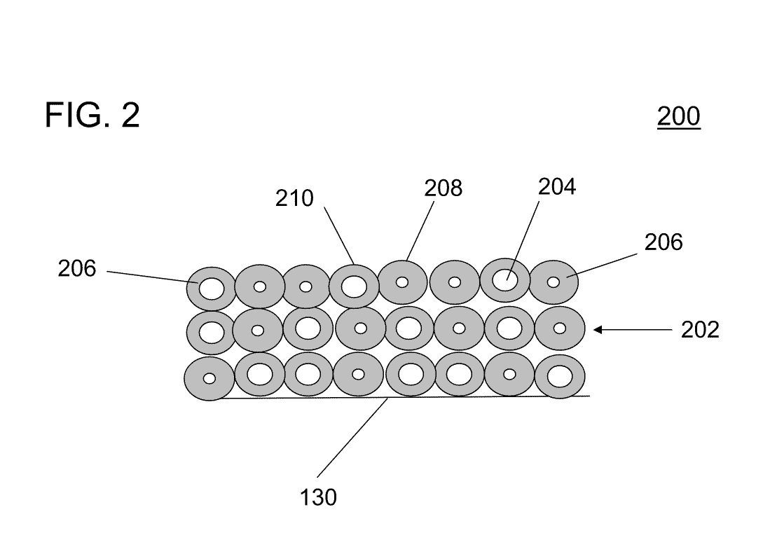

In one embodiment of the invention, at least a portion of the surface of stent framework 130 or the external surface of balloon 112 is overlaid with a coating composition 200 that includes layers of microspheres 202, as shown in FIG. 2. Depending on the nature of the therapeutic agent to be delivered, the coating may be present on the interior (luminal) surface of the stent, the exterior surface of the stent, both stent surfaces, or the exterior surface of balloon 112. In some embodiments, microspheres 202 include nanospheres having a diameter of less than 100 nanometers; in other embodiments, microspheres 202 range in size from 50 μm to approximately 500 μm. The size of the microspheres is selected so that the diameter of the largest microspheres is less than 10% of the diameter of the stent strut to be coated. For example, if the diameter of the stent strut is 250 μm, the preferred microsphere size is smaller than approximately 25 μm. In one embodiment, medical devices such as the surface of balloon 112, having a large surface area may include microspheres 202 having a diameter of a few hundred micrometers.

In one embodiment, the microspheres are hollow, and have an internal chamber 204 that is surrounded by wall 206. The therapeutic agent to be delivered is sequestered within chamber 204. Wall 206 comprises a biodegradable material that begins to erode soon after delivery of the stent to the treatment site. When the integrity of wall 206 is breeched, the therapeutic agent contained within chamber 204 is released. Consequently, the time of onset of delivery of the therapeutic agent from each microsphere depends on the thickness of wall 206. The thickness of wall 206 of the microspheres ranges from a few nanometers to hundreds of micrometers, and makes up between 1% and 99% of the diameter of the microsphere. In one embodiment, the wall thicknesses of the microspheres within stent coating 200 are variable, and form at least two populations of microspheres: those having thick walls 208 and those having thin walls, 210. Consequently, the rate of delivery of the therapeutic agent is determined by the ratio of wall thicknesses of microspheres 202. The proportion of thick and thin walled microspheres can be adjusted to provide release of the therapeutic agent in a desired, predetermined elution profile.

Therapeutic agent release is proportional to the surface area of coating 200, and because coating 200 approximates a concentric tubular surface overlaying the surface of the stent framework 130, as coating 200 erodes and becomes thinner, the surface area for therapeutic agent release becomes smaller as it approaches the surface of stent framework 130. In one embodiment, microspheres 202 are arranged in layers within coating 200. Microspheres 206 are varied within each layer so that a different proportion of thick-walled microspheres 208 and thin walled microspheres 210 are present in each layer. In one embodiment the proportion of thin walled microspheres 210 is increased in layers near the surface of stent framework 130, where the surface area of coating 200 is smallest, and decreased in the outer layers of coating 200 where the delivery surface is larger. In one embodiment, the proportion of thin walled microspheres is increased in each layer to provide a constant (zero order) rate of release of therapeutic agent throughout the entire time of delivery at the treatment site. In another embodiment, there is a preponderance of thin-walled microspheres 210 in the outer layer of coating 200, causing a burst of therapeutic agent release soon after placement of the stent, followed by a steady rate of release from inner layers. In yet another embodiment, there is a preponderance of thick-walled microspheres 210 in the outer layer of coating 200, delaying the release of therapeutic agent from the microspheres in the inner layer(s) of coating 200.

One or more therapeutic agents are sequestered within microspheres 202. Various therapeutic agents, such as anticoagulants, antiinflammatories, fibrinolytics, antiproliferatives, antibiotics, therapeutic proteins or peptides, DNA, and recombinant DNA products, or other bioactive agents, diagnostic agents, radioactive isotopes, or radiopaque substances may be used depending on the anticipated needs of the targeted patient population. The formulation containing the therapeutic agent may additionally contain excipients including solvents or other solubilizers, stabilizers, suspending agents, antioxidants, and preservatives, as needed to deliver an effective dose of the therapeutic agent to the treatment site.

In one embodiment, the walls of microspheres 202 comprise one or more of a variety of biocompatible materials such as hydrogels, thermosensitive polymers, such as poly N-isopropylacrylamide (PNIPAM), synthetic biodegradable polymers, such as polylactic acid and its copolymers, polyamide esters, polyvinyl esters, polyvinyl alcohol, polyanhydrides, natural biodegradable polymers, such as polysaccharides; enzymatically degradable polymers, such as proteins, collagen, poly-L-glutamic acid, elastin, albumin, fibrin, hyaluronic acid, chitosan, and alginic acid; bioactive glasses, such as Bioglass® 45S5 glass; biodegradable calcium phosphates, such as β-tricalcium phosphates; liposomes, vesicles, and any other appropriate material. These materials may be used alone or in various combinations to give the microspheres unique properties such as controlled rates of degradation, and to provide the desired time of onset and rate of delivery of the therapeutic agent to be delivered at the treatment site.

The microspheres are affixed to the surface of stent framework 130 or balloon 112 using natural or synthetic biodegradable adhesives such as hydrocolloids, acrylic-based adhesives, fibrin or collagen glues, or any other appropriate means known in the art.

In one embodiment, a protective surface coating (cap coat) is placed over coating 200. This protective coating comprises one or more biocompatible, biodegradable polymers such as hyaluronic acid, polylactic acid, polyglycolic acid, or their copolymers. Such a coating prevents loss of or damage to coating 200 during handling and delivery of the stent. Once in place at the treatment site, the protective coating degrades and allows delivery of the therapeutic agent from the microspheres.

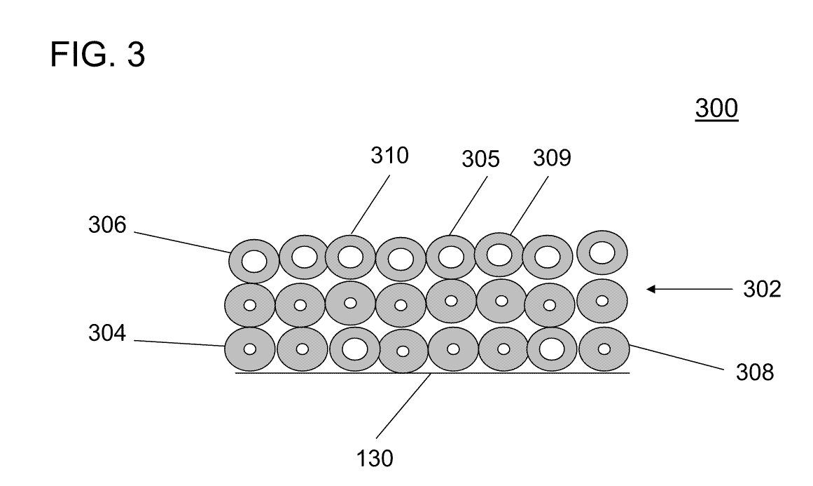

In one embodiment, coating 300 includes microspheres having variable wall thicknesses, and comprising different materials, as shown in FIG. 3. Microspheres 302 comprising a first material 305, are either thick-walled, 304, or thin-walled, 306. Similarly, microspheres 302 comprising a second material 309 are thick-walled, 308, or thin-walled, 310. The four different groups of microspheres 302 are combined to provide the desired rate and duration of therapeutic agent delivery. In one embodiment, the first material 305 erodes slowly, and the second material 309 erodes quickly after stent placement. Therefore, therapeutic agent is delivered, first from thin-walled microspheres 310 (second material), followed by therapeutic agent delivered from thin-walled microspheres 306 (first material), next from thick-walled microspheres 308 (second material) and finally, thick-walled microspheres 304 (first material). Microspheres from each group are arranged in layers to provide the desired therapeutic agent elution profile. For example, as shown in FIG. 3, the outer layer comprises thin-walled microspheres 306, 310 of both first material 305 and second material 309, respectively, to provide initial, rapid release of therapeutic agent after placement of stent 120. This is followed by an intermediate layer of thick-walled microspheres 304, 308 of both first material 305 and second material 309, providing a prolonged period of steady release of therapeutic agent. Finally, an inner layer disposed directly on the surface of stent framework 130, comprises thick-walled microspheres 308 and 304, and some thin-walled microspheres 306 composed of the first material. Inclusion of microspheres 306 in the inner most layer increases the rate of therapeutic agent release to accommodate the decreased surface area of coating 302 near the surface of stent framework 130, and maintains a steady (zero order) rate of therapeutic agent delivery following the initial burst of the outer most layer.

In another embodiment, two therapeutics agents are delivered simultaneously from coating 300. For example, an anti-inflammatory drug such as dexamethasone, may be delivered in the initial burst of delivery from the outer layer, followed by delivery of an antiproliferative drug (zotarolimus, for example) in a slower, steady release from the inner layers. In this instance, the composition and geometry (thick- or thin-wall) of the microspheres 302 in each layer of coating 300 can be modified to provide the desired elution profile of each drug independently of the other drug. In yet another embodiment, a coating 300 having one composition is overlaid on the exterior surface and another on the luminal surface of stent framework 130, allowing simultaneous delivery of one therapeutic agent, an anticoagulant, for example, from the luminal surface, and a second therapeutic agent such as an antiproliferative from the external surface, each at an optimal delivery rate. In another embodiment, a coating on the surface of the balloon provides rapid delivery of one therapeutic agent, and is paired with a second coating on one or more stent surfaces that provides prolonged delivery of the same or a different therapeutic agent.

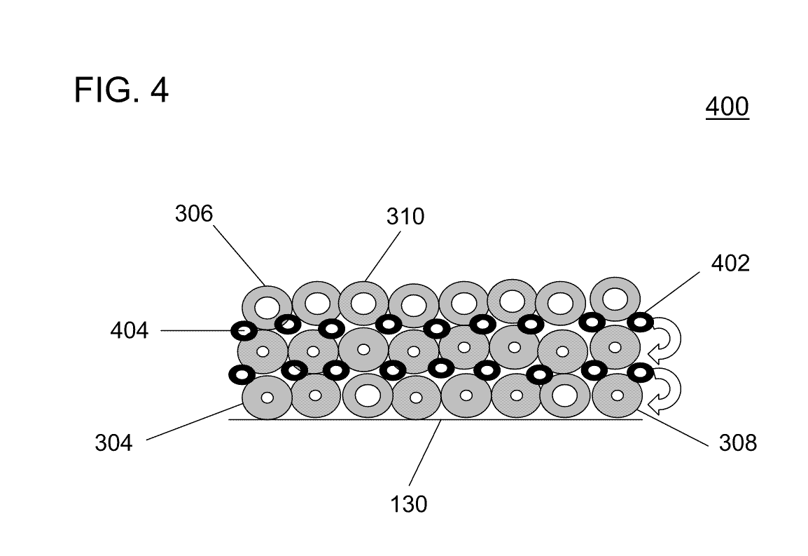

In one embodiment, shown in FIG. 4, one or more degradative agents are incorporated into coating 400 that facilitate the break-down of at least some of the therapeutic agent containing microspheres comprising the coating. In one embodiment, these degradative agents are sequestered within small degradation microspheres 402 so that they do not act on the surrounding therapeutic agent containing microspheres until they are released. In one embodiment degradation microspheres 402 comprise hyaluronic acid or gelatin, which is stable under dry conditions, for example during manufacture, storage and delivery of stent 120. However, after placement of stent 120, coating 400 is bathed in bodily fluids, and the hyaluronic acid or gelatin containing walls of degradation microspheres 402 erode and release the degradative agent from inner chamber 404. In one embodiment, degradation microspheres 402 are relatively small compared to therapeutic agent containing microspheres 304, 306, 308 or 310. Due to their high surface-to-volume ratio, degradation microspheres 402 degrade rapidly, and shortly after delivery of stent 120, degradation microspheres 402 in the outer layers of coating 400 release the degradative agent. As shown by the block arrows in FIG. 4, the degradative agent acts on the next layer of microspheres, by accelerating the degradation of the microspheres and release of the therapeutic agent from the microspheres.

In one exemplary embodiment, the walls of microspheres 308 and 310 comprise dextrin and the therapeutic agent to be delivered by microspheres 308 and 310 is an anti-inflammatory such as dexamethasone. The walls of microspheres 304 and 306 comprise biodegradable poly-lactic acid, and the therapeutic agent to be delivered by microspheres 304 and 306 is an antiproliferative such as zotarolimus. The walls of degradation microspheres 402 comprise hyaluronic acid and the degradative agent within microspheres 402 is α-amylase enzyme. Soon after delivery of stent 120, the hyaluronic acid walls of degradation microspheres 402 are eroded in the aqueous environment, and the α-amylase enzyme is released in proximity to microspheres 308 and 310. The α-amylase enzyme, in turn, increases the rate of degradation of the walls of microspheres 308 and 310, and thereby increases the rate of dexamethasone release. In contrast, the rate of release of zotarolimus is determined only by the concentration of microspheres 304 and 306 in each layer of coating 400 and the wall thicknesses of each group of microspheres 304 and 306. This and similar embodiments demonstrate the range of therapeutically effective delivery systems for a wide array of therapeutic agents that can be devised using microspheres of variable wall thicknesses, in accordance with the invention.

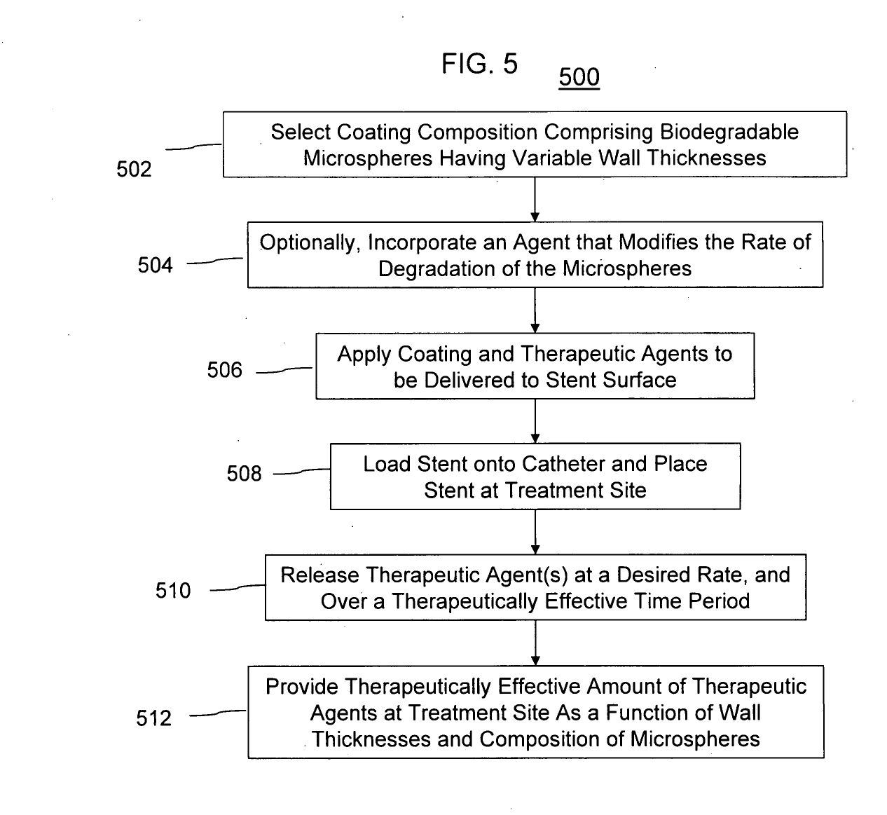

FIG. 5 is a flowchart of method 500 for treating a vascular condition using a stent having a coating comprising microspheres, in accordance with the present invention. The method includes selecting biodegradable microspheres having variable wall thicknesses, as indicated in Block 502. One or more therapeutic agents to be delivered are sequestered within a central chamber of the microspheres surrounded by the microsphere wall. The wall of the microsphere comprises one or more biocompatible, biodegradable materials that degrade and are removed after placement of the stent, allowing release of the therapeutic agent at the treatment site. The rate of release of the therapeutic agent depends on the composition and thickness of the microsphere walls.

Optionally, a degradative agent that breaks down the microsphere walls may be incorporated within the coating to modify the rate of degradation of the microsphere walls and therefore therapeutic agent delivery, as indicated in Block 504. In some embodiments, the degradative agent is kept dry, and therefore inactive until exposed to bodily fluids after delivery. In other embodiments, the degradative agent is incorporated into degradation microspheres that degrade soon after placement of stent 120.

The microspheres are applied to the surface of stent framework 130 in the form of a coating that may include polymers and adhesives that cause the coating to adhere to stent framework 130 in smooth, thin layers, as indicated in Block 506. In some embodiments, the coating comprises layers of microspheres having differing wall thicknesses, and in some cases different compositions. Next, as indicated in Block 508, the coated stent 120 is mounted on a catheter and delivered to the treatment site. At the treatment site, the stent is positioned across the lesion to be treated and expanded. The catheter is then withdrawn from the body. Once positioned at the treatment site, the stent provides support for the vessel wall and therapeutic agent delivery at a therapeutically effective rate for a defined time period after placement of the stent (Block 510). The coating delivers a therapeutically effective amount of therapeutic agent at the treatment site as a function of the wall thicknesses and compositions of the microspheres within the coating, as indicated in Block 512.

While the invention has been described with reference to particular embodiments, it will be understood by one skilled in the art that variations and modifications may be made in form and detail without departing from the spirit and scope of the invention.

1. A system for treating a vascular condition comprising:
a catheter;
a medical device disposed on the catheter;
a therapeutic agent coating disposed on at least a portion of the medical device, the therapeutic agent coating including a first plurality of biodegradable microspheres having a first wall thickness and a second plurality of biodegradable microspheres having a second wall thickness, the biodegradable microspheres including at least one therapeutic agent, wherein a release rate of the at least one therapeutic agent is determined based on the first wall thickness and the second wall thickness.

2. The system of claim 1 wherein the first wall thickness is greater than the second wall thickness and wherein the first plurality of biodegradable microspheres includes a first therapeutic agent and the second plurality of biodegradable microspheres includes a second therapeutic agent.

3. The system of claim 1 wherein the first plurality of biodegradable microspheres comprises a first material and the second plurality of biodegradable microspheres comprises a second material.

4. The system of claim 2 wherein the first material degrades at a first rate and the second material degrades at a second rate.

5. The system of claim 2 further comprising a third plurality of biodegradable microspheres having a third wall thickness and including a third therapeutic agent.

6. The system of claim 5 wherein the third wall thickness is substantially equal to the first wall thickness and wherein the third therapeutic agent is the same as the second therapeutic agent.

7. The system of claim 6 further comprising a fourth plurality of biodegradable microspheres having a fourth wall thickness and including a fourth therapeutic agent.

8. The system of claim 7 wherein the fourth wall thickness is substantially equal to the second wall thickness and wherein the fourth therapeutic agent is the same as the first therapeutic agent.

9. The system of claim 8 wherein the first plurality of biodegradable microspheres and the second plurality of biodegradable microspheres comprise a first material and wherein the third plurality of biodegradable microspheres and the fourth plurality of biodegradable microspheres comprise a second material.

10. The system of claim 9 wherein the first material degrades at a first rate and the second material degrades at a second rate.

11. The system of claim 1 wherein the therapeutic agent coating comprises a plurality of layers of biodegradable microspheres and the release rate of the at least one therapeutic agent is determined based on a distance the layer is from the stent surface.

12. The system of claim 1 wherein the therapeutic agent coating further comprises a plurality of biodegradable degradation microspheres including a degradative agent wherein the degradative agent modifies the degradation rate of at least one of the first plurality of biodegradable microspheres and the second plurality of biodegradable microspheres.

13. The system of claim 1 wherein at least a portion of the biodegradable microspheres are nanospheres having a diameter of less than 100 nanometers.

14. The system of claim 1 further comprising a cap coat disposed on an outer surface of the therapeutic agent coating.

15. A medical device for treating a vascular condition, the device comprising:
a coating disposed on a surface of the device, the coating comprising a plurality of biodegradable microspheres of variable wall thicknesses, wherein the rate of release of at least one therapeutic agent from the coating is dependent on the wall thicknesses of the biodegradable microspheres.

16. The device of claim 15 wherein the coating comprises a plurality of layers of biodegradable microspheres, wherein a release rate of the at least one therapeutic agent is determined as a function of a distance that a layer is from the device surface.

17. The device of claim 15 further comprising a first plurality of biodegradable microspheres having a first composition and a second plurality of biodegradable microspheres having a second composition wherein the release rate of the at least one therapeutic agent is dependent on the first composition and the second composition.

18. The device of claim 17 wherein a first portion of the first plurality of biodegradable microspheres and a first portion of the second plurality of biodegradable microspheres have a first wall thickness and wherein a second portion of the first plurality of biodegradable microspheres and a second portion of the second plurality of biodegradable microspheres have a second wall thickness, the first wall thickness greater than the second wall thickness.

19. The device of claim 18 wherein the first plurality of biodegradable microspheres includes a first therapeutic agent and the second plurality of biodegradable microspheres includes a second therapeutic agent.

20. The device of claim 16 wherein the coating further comprises a plurality of biodegradable degradation microspheres including a degradative agent wherein the degradative agent modifies the degradation rate of at least one of the first plurality of biodegradable microspheres and the second plurality of biodegradable microspheres.
